(19) United States
(12) Patent Application Publication
(10) Pub. No.: US 2003/0161803 A1
(43) Pub. Date: Aug. 28, 2003

(54) Cosmetic compositions comprising a cosmetic active agent and an exogenous ligand-receptor system for the hair, and hair treatment process using these compositions

(76) Inventors: Gabin Vic, Compiegne (FR); Aude Livoreil, Paris (FR); Bruno Bernard, Neuilly Sur Seine (FR)

Correspondence Address: D. Douglas Price, Steptoe & Johnson, Box PTO, 1330 Connecticut Avenue, NW, Washington, DC, 20036, US

(21) Appl. No.: 10/330,481

(22) Filed: Dec. 30, 2002

(30) Foreign Application Priority Data
Dec. 31, 2001 (FR) 01 17074

(51) Int. Cl. A61K 7/06; A61K 7/00; A61K 7/13; A61K 31/4188

(52) U.S. Cl. 424/70.1; 514/393; 8/406

(57) ABSTRACT

The present invention relates to cosmetic compositions comprising a biotin derivative and/or an agent for complexing this derivative and, covalently bonded to one of these two compounds or to both of them, at least one active agent other than dyes.

The present invention also relates to a process for treating the hair based on these compositions, which is directed towards creating on the fibres a deposit of material that provides novel cosmetic properties.

The present invention relates to a cosmetic composition comprising at least one biotin compound (modified or unmodified biotin) bearing at least one group with non-colouring cosmetic activity.

The present invention also relates to a cosmetic composition comprising at least one agent for complexing modified or unmodified biotin, bearing at least one group with non-colouring cosmetic activity.

The present invention also relates to a cosmetic composition comprising modified or unmodified biotin combined with at least one complexing agent, the biotin or the complexing agent bearing at least one group with non-colouring cosmetic activity.

The present invention also relates to several hair treatment processes based on these compounds, which are directed towards creating on the fibre a deposit of material that provides novel cosmetic properties.

It is advantageous to be able to fix onto keratin materials, and in particular the hair, active agents that provide long-lasting benefit. Direct chemical grafting is one possible solution for achieving this objective. Unfortunately, these chemical grafting techniques pose problems of harmfulness or result in degradation of the integrity of the keratin materials. There is thus still a need to create a deposit of material on the hair that is remnant with respect to shampooing, that does not degrade the hair fibre, that can be removed as desired and that is optionally crosslinkable.

Biotin, also known as vitamin H or B8, is a water-soluble vitamin consisting of an imidazoline nucleus and a tetrahydrothiophene ring bearing a side chain containing five carbon atoms. Biotin is a nutritive element for the hair and the skin. Biotin binds naturally and with very great affinity to avidin, a glycoprotein found in raw egg white. Biotin and avidin together form a complex by non-covalent bonding that is among the most stable existing between biological molecules, with a dissociation constant of 1.3×10⁻¹⁵ at pH 5. Furthermore, avidin has four sites of interaction for biotin.

The Applicant has discovered that by using the forces of association between two exogenous compounds for the hair, cosmetic active agents (prebound to at least one of the compounds) may be deposited on the surface of the hair.

A first subject of the invention is thus a composition, especially a cosmetic composition, comprising at least one biotin compound (modified or unmodified biotin) bearing at least one group with non-colouring cosmetic activity.

A second subject of the invention is a composition, especially a cosmetic composition, comprising at least one compound capable of forming with a biotin compound, optionally bearing at least one group with non-colouring cosmetic activity, a complex whose dissociation constant is less than or equal to 10⁻¹ at a temperature of 25° C., the said completing compound bearing at least one group with non-colouring cosmetic activity.

A third subject of the invention is thus a composition, especially a cosmetic composition, comprising:

a biotin compound a) chosen from biotin and modified biotins,

a compound b) capable of forming with compound a) a complex whose dissociation constant is less than or equal to 10⁻¹ at a temperature of 25° C., at least one of these compounds a) or b) bearing at least one group with non-colouring cosmetic activity.

Another subject of the invention is all the cosmetic processes for treating keratin materials using these compositions or the constituents a) and b) of the third subject of the invention.

Other subjects of the present patent application will emerge on reading the description and the examples that follow.

The compounds a), i.e. biotin and modified biotins, are preferably chosen from the compounds of formula (I)

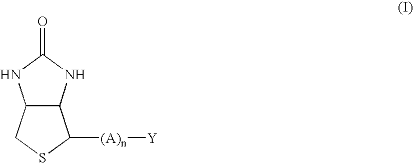

with A denoting a linear or branched, saturated or unsaturated divalent radical containing from 1 to 100 carbon atoms, optionally interrupted with one or more hetero atoms and optionally substituted with one or more groups chosen from hydroxyl, amino, halogen, aryl, phosphate, phosphorate, sulphate, sulphonate, carboxyl, alkoxycarbonyl and alkoxy groups,

with Y denoting a group Z chosen from carboxyl, mercaptan, alkoxycarbonyl, amino, ammonio or a radical BW, W being a residue with cosmetic activity and B being a species for fixing the residue W to the biotin-based skeleton,

with n denoting 0 or 1,

with B denoting, for example, an amine, imine, amide, ester, disulphide, thioester, urethane, urea, ether, thioether, azo, methine or epoxy group, an aromatic or heterocyclic residue or a dialkylpolysiloxane residue,

with W possibly being derived from UV-screening molecules, hydrating or emollient molecules, conditioners, antistatic agents, antiperspirants, fragrancing materials, reducing molecules, oxidizing molecules, antimicrobial agents, antidandruff agents, mineral or organic particulates that may be in the form of nanotubes or other three-dimensional assemblies, for instance fullerenes (e.g. C60-C70 fullerenes) onto which are optionally adsorbed one or more polymers, and anionic or nonionic, amphoteric or cationic film-forming agents onto which are optionally adsorbed organic or mineral particles.

The term “particles” covers mineral particles and organic particles. The mineral particles may consist of the oxides, oxide dihydrates, hydroxides, carbonates, sulphides, silicates and sulphates of silicon, of calcium, of magnesium, of zinc, of aluminium, of titanium, of zirconium or of cerium, nacres, micas, particles consisting of native metals, i.e. alkali metals, alkaline-earth metals, transition metals or rare-earth metals, which are optionally coated, and alloys thereof. Among the metals that are preferred are aluminium, copper, silver, gold, indium, iron, platinum, nickel, molybdenum, silicon, titanium, tungsten, antimony, palladium, cadmium, zinc, tin and selenium, and alloys thereof. Among the above list, gold, silver, palladium and platinum and alloys thereof are preferred, silver and gold being the preferred metals.

The organic particles may consist of latices, polystyrene, polystyrene derivatives, silicones, fluoro polymers, polypropylene, polyethylene, poly(meth)acrylic acid, polymethacrylate, polyurethane, polyamide, polycarbonate, polyvinyl chloride, polyvinyl acetate, fluoropolymers, polyethylenes, polypropylene, polyisobutylene, poly(1-butylene), copolymers and blends of the polymers mentioned, alkyl cellulose, hydroxyalkyl cellulose, cellulose ethers, cellulose esters, hydroxypropylcellulose, hydroxypropyldextran, hydroxypropylmethyl cellulose, cellulose acetate, carboxyethylcellulose, cellulose sulphate, dextran sulphate, polyvinyl alcohol, polyethylene oxide, polyvinyl chloride and polyvinylpyrrolidone.

The polystyrene particles are commercially available from Polyscience Inc. (Warrington, Pa.) or Duke Scientific Corporation (Palo Alto, Calif.).

These organic particles may be crosslinked with crosslinking agents such as, for example, divinylbenzene, glutaraldehyde, 1,4-bis(acryloyl)piperazine, carbodiimides, N-hydroxysuccinimide and derivatives, divinyl sulphone, dithiobis(succinimidyl)propioniate and N-succinimidyl-3-(2-pyridyldithio)propionate.

The term “particles” means particles that are between 1 nm and 100 μm in size, preferably between 1 nm and 1000 nm in size and even more preferably between 1 nm and 50 nm in size. The term “particle size” means the maximum size it is possible to measure between two diametrically opposite points of the particle.

The particles according to the invention may be of spherical, lamellar or fibrillar shape, or of totally random shape.

The biotin derivatives that may be used according to the invention are available, for example, from Sigma-Aldrich or Carbomer Inc. and may be chosen from bovine albumin-biotin, bovine albumin-biotin-gold labelled (5 or 20 nm), biotin-dextran, biotin-fluorescein, latex-biotin, biotin-carboxymethylcellulose, biotin-chitosan, biotinamidocaproyl peroxidase, biotinamidocaproyl alkaline phosphatase, biotin-agarose, concanavalin A-biotin, β-galactosidase-biotin, lectin-biotin conjugates and B-phycoerythrin-biotin.

The group W may be derived from the following active agents:

modified or unmodified, optionally hydrolysed saccharides, oligosaccharides and polysaccharides,

amino acids, oligopeptides, peptides, modified or unmodified, optionally hydrolysed proteins, polyamino acids and enzymes,

branched or unbranched fatty alcohols and fatty acids,

animal, plant or mineral waxes,

ceramides and pseudoceramides,

hydroxylated organic acids,

UV screening agents,

antioxidants and free-radical scavengers,

chelating agents,

antidandruff agents,

seborrhoea regulators,

calmants,

cationic surfactants,

cationic or amphoteric polymers,

optionally organomodified silicones,

mineral, plant or animal oils,

polyisobutenes and poly(α-olefins),

esters,

soluble or dispersed anionic polymers,

soluble or dispersed nonionic polymers,

reducing agents,

and mixtures thereof.

In general, the compounds of modified or unmodified, optionally hydrolysed saccharide, oligosaccharide and polysaccharide type, which may be used in the present invention, are chosen from those described especially in “Encyclopedia of Chemical Technology”, Kirk-Othmer, Third Edition, 1982, volume 3, pp. 896-900 and volume 15, pp. 439-458, in “Polymers in Nature” by E. A. MacGregor and C. T. Greenwood, published by John Wiley & Sons, Chapter 6, pp. 240-328, 1980, and in “Industrial Gums—Polysaccharides and their Derivatives”, edited by Roy L. Whistler, Second Edition, published by Academic Press Inc., the content of these three publications being included in their entirety in the present patent application by reference.

As examples of modified or unmodified, optionally hydrolysed saccharides, oligosaccharides and polysaccharides that may be used in the invention, mention may be made especially of glucans, modified or unmodified starches (such as those derived, for example, from cereals, for instance wheat, corn or rice, from vegetables, for instance haricot beans, from tubers, for instance potato or cassaya), which are different from the starch betain as described above, amylose, amylopectin, glycogen, dextrans, β-glucans, celluloses and derivatives thereof (methylcelluloses, hydroxyalkylcelluloses, ethylhydroxyethylcelluloses or carboxymethylcelluloses), fructosans, inulin, levane, mannans, xylans, lignins, arabans, galactans, galacturonans, chitin, glucoronoxylans, arabinoxylans, xyloglucans, galactomannans, glucomannans, pectic acids and pectins, alginic acid and alginates, arabinogalactans, carrageenans, agars, glycosaminoglucans, gum arabics, gum tragacanths, ghatti gums, karaya gums, carob gums, guar gums and xanthan gums.

Examples of amino acids that may be mentioned include cysteine, lysine, alanine, N-phenylalanine, arginine, glycine and leucine, and mixtures thereof. As modified or unmodified, optionally hydrolysed oligopeptides, peptides and proteins that may be used according to the invention, mention may be made especially of modified or unmodified wool or silk protein hydrolysates, and plant proteins such as wheat proteins.

Among the polyamino acids that may be used, mention may be made of polylysine.

Among the enzymes that may be used, mention may be made of laccases, peroxidases, lipases, proteases, glycosidases, dextranases, uricases and alkaline phosphatase.

Among the branched or unbranched fatty acids that are suitable in the present invention, mention may be made especially of C₈-C₃₀ carboxylic acids such as palmitic acid, oleic acid, linoleic acid, myristic acid, stearic acid and lauric acid, and mixtures thereof. The fatty alcohols that may be used in the present invention especially include C₈-C₃₀ alcohols such as, for example, palmityl alcohol, oleyl alcohol, linoleyl alcohol, myristyl alcohol, stearyl alcohol and lauryl alcohol.

For the purposes of the present invention, a wax is a lipophilic compound that is solid at room temperature (about 25° C.), with a reversible solid/liquid change of state, having a melting point of greater than about 40° C. and which may be up to 200° C., and having an anisotropic crystal organization in the solid state. In general, the size of the wax crystals is such that crystals scatter and/or diffuse light, giving the composition comprising them a more or less opaque, cloudy appearance. By raising the wax to its melting point, it is possible to make it miscible with oils and to form a microscopically homogeneous mixture, but on returning the temperature of the mixture to room temperature, a microscopically and macroscopically detectable recrystallization of the wax in the oils of the mixture is obtained (opalescence).

As waxes that may be used in the present invention, mention may be made of waxes of animal origin such as beeswax, spermaceti, lanolin wax and lanolin derivatives; plant waxes such as carnauba wax, candelilla wax, ouricury wax, Japan wax, cocoa butter, cork fibre wax or sugar cane wax; mineral waxes, for example paraffin wax, petroleum jelly wax, lignite wax, microcrystalline waxes or ozokerites.

Among the ceramides that may be mentioned in particular are the ceramides of categories I, II, III and V according to the Downing classification, and more particularly N-oleyldehydrosphingosine.

The hydroxylated organic acids are chosen from those that are well known and used in the art. Mention may be made especially of citric acid, lactic acid, tartaric acid and malic acid.

The UV-A-active and/or UV-B-active sunscreens that may be used according to the invention are those that are well known to those skilled in the art. Mention may be made especially of dibenzoylmethane derivatives such as 4-methyldibenzoylmethane, 4-isopropyldibenzoylmethane, 4-tert-butyldibenzoylmethane, 2,4-dimethyldibenzoylmethane, 4-tert-butyl-4′-diisopropyldibenzoylmethane, p-aminobenzoic acid and its esters such as 2-ethylhexyl p-dimethylaminobenzoate and N-propoxylated ethyl p-aminobenzoate, salicylates such as triethanolamine salicylate, cinnamic acid esters such as 2-ethylhexyl 4-methoxycinnamate, methyl diisopropylcinnamate, methyl anthranilate, benzotriazole derivatives, triazine derivatives, β,β′-diphenylacrylate derivatives such as 2-ethylhexyl 2-cyano-3,3-diphenylacrylate and ethyl 2-cyano-3,3-diphenylacrylate, 2-phenylbenzimidazole-5-sulphonic acid and its salts, benzophenone derivatives, benzylidenecamphor derivatives, silicone screening agents, etc.

As examples of antioxidants and free-radical scavengers that may be used in the present invention, mention may be made of ascorbic acid, ascorbyl compounds such as ascorbyl dipalmitate, t-butylhydroquinone, polyphenols such as phloroglucinol, sodium sulphite, erythorbic acid and flavonoids.

The chelating agents may be chosen especially from EDTA (ethylenediaminetetraacetic acid) and its salts such as disodium EDTA and dipotassium EDTA, phosphate compounds such as sodium metaphosphate, sodium hexametaphosphate and tetrapotassium pyrophosphate, and phosphonic acids and salts thereof, such as ethylenediaminetetramethylenephosphonic acid salts.

The antidandruff agents are chosen, for example, from:

benzethonium chloride, benzalkonium chloride, chlorhexidine, chloramine T, chloramine B, 1,3-dibromo-5,5-dimethylhydantoin, 1,3-dichloro-5,5-dimethylhydantoin, 3-bromo-1-chloro-5,5-dimethylhydantoin and N-chlorosuccinimide;

1-hydroxy-2-pyridone derivatives such as, for example, 1-hydroxy-4-methyl-2-pyridone, 1-hydroxy-6-methyl-2-pyridone and 1-hydroxy-4,6-dimethyl-2-pyridone;

trihalocarbamides;

triclosan;

azole compounds such as climbazole, ketoconazole, clotrinazole, econazole, isoconazole and miconazole b;

antifungal polymers such as amphotericin B or nystatin;

selenium sulphides;

sulphur in its various forms, cadmium sulphide, allantoin, coal tar or wood tar and in particular derivatives thereof, cade oil, undecylenic acid, fumaric acid and allylamines such as terbinafine.

The said agents may also be used in the form of the addition salts thereof with physiologically acceptable acids, especially in the form of the sulphuric acid, nitric acid, thiocyanic acid, hydrochloric acid, hydrobromic acid, hydriodic acid, phosphoric acid, acetic acid, benzoic acid, glycolic acid, aceturic acid, succinic acid, nicotinic acid, tartaric acid, maleic acid, palmitic acid, methanesulphonic acid, propanoic acid, 2-oxopropanoic acid, propanedioic acid, 2-hydroxy-1,4-butanedioic acid, 3-phenyl-2-propenoic acid, α-hydroxybenzeneacetic acid, ethanesulphonic acid, 2-hydroxyethanesulphonic acid, 4-methylbenzenesulphonic acid, 4-amino-2-hydroxybenzoic acid, 2-phenoxybenzoic acid, 2-acetyloxybenzoic acid, picric acid, lactic acid, citric acid, malic acid, oxalic acid and amino acid salts.

The antidandruff agents mentioned above may also, where appropriate, be used in the form of the addition salts thereof with physiologically acceptable organic or mineral bases. Examples of organic bases are especially low molecular weight alkanolamines such as ethanolamine, diethanolamine, N-ethylethanolamine, triethanolamine, diethylaminoethanol and 2-amino-2-methylpropanedione; non-volatile bases such as ethylenediamine, hexamethylenediamine, cyclohexylamine, benzylamine and N-methylpiperazine; quaternary ammonium hydroxides, for example trimethylbenzyl hydroxide; guanidine and its derivatives, and particularly its alkyl derivatives Examples of mineral bases are especially the alkali metal salts, for instance the sodium or potassium salts; the ammonium salts, the alkaline-earth metal salts, for instance the magnesium or calcium salts; the salts of cationic di-, tri- or tetravalent metals, for instance the zinc, aluminium and zirconium salts. Alkanolamines, ethylenediamine and mineral bases such as the alkali metal salts are preferred.

The seborrhoea regulators are, for example, succinylchitosan and poly-β-alanine.

The calmants are, for example, azulene and glycyrrhetinic acid.

The cationic surfactants are those that are well known per se, such as optionally polyoxyalkylenated primary, secondary or tertiary fatty amine salts; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives.

The term “cationic polymer” means any polymer containing cationic groups and/or groups that may be ionized into cationic groups.

The cationic polymers that may be used in accordance with the present invention may be chosen from any of those already known per se as improving the cosmetic properties of hair treated with detergent compositions, i.e. especially those described in patent application EP-A-0 337 354 and in French patent applications FR-A-2 270 846, 2 383 660, 2 598 611, 2 470 596 and 2 519 863.

The preferred cationic polymers are chosen from those containing units comprising primary, secondary, tertiary and/or quaternary amine groups that may either form part of the main polymer chain or may be borne by a side substituent that is directly attached thereto.

The cationic polymers used generally have a number-average molecular mass of between 500 and 5×10⁶ approximately and preferably between 10³ and 3×10⁶ approximately.

Among the cationic polymers that may be mentioned more particularly are polymers of the polyamine, polyamino amide and polyquaternary ammonium type. These are known products.

The polymers of the polyamine, polyamino amide and polyquaternary ammonium type that may be used in the composition of the present invention are those described in French patents 2 505 348 and 2 542 997. Among these polymers, mention may be made of:

(1) homopolymers or copolymers derived from acrylic acid or methacrylic acid esters or amides;

(2) the cellulose ether derivatives containing quaternary ammonium groups, described in French patent 1 492 597;

(3) cationic cellulose derivatives such as cellulose copolymers or cellulose derivatives grafted with a water-soluble quaternary ammonium monomer, and described in particular in U.S. Pat. No. 4,131,576, such as hydroxyalkylcelluloses, for instance hydroxymethyl-, hydroxyethyl- or hydroxypropylcelluloses grafted, in particular, with a methacryloylethyltrimethylammonium, methacrylamidopropyltrimethylammonium or dimethyldiallylammonium salt;

(4) the cationic polysaccharides described more particularly in U.S. Pat. Nos. 3,589,578 and 4,031,307, such as guar gums containing cationic trialkylammonium groups;

(5) polymers consisting of piperazinyl units and of divalent alkylene or hydroxyalkylene groups containing straight or branched chains, optionally interrupted by oxygen, sulphur or nitrogen atoms or by aromatic or heterocyclic rings, as well as the oxidation and/or quaternization products of these polymers. Such polymers are described, in particular, in French patents 2 162 025 and 2 280 361;

(6) water-soluble polyamino amides such as those described, in particular, in French patents 2 252 840 and 2 368 508;

(7) polyamino amide derivatives, for example, adipic acid/dialkylaminohydroxyalkyldialkylenetriamine polymers in which the alkyl group contains from 1 to 4 carbon atoms and preferably denotes a methyl, ethyl or propyl group, and the alkylene group contains from 1 to 4 carbon atoms and preferably denotes an ethylene group. Such polymers are described in particular in French patent 1 583 363.

(8) polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyamino amide resulting therefrom is reacted with epichlorohydrin in a molar ratio of epichlorohydrin relative to the secondary amine group of the polyamino amide of between 0.5:1 and 1.8:1. Such polymers are described in particular in U.S. Pat. Nos. 3,227,615 and 2,961,347;

(9) cyclopolymers of alkyldiallylamine or of dialkyldiallylammonium such as the dimethyldiallylammonium chloride homopolymer and copolymers of diallyldimethylammonium chloride and of acrylamide;

(10) quaternary diammonium polymers with a number-average molecular mass generally of between 1000 and 100 000 such as those described, for example, in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020;

(11) polyquaternary ammonium polymers such as those especially described in patent application EP-A-122 324;

(12) quaternary polymers of vinylpyrrolidone and of vinylimidazole, such as, for example, the products sold under the names Luviquat® FC 905, FC 550 and FC 370 by the company BASF;

(13) polyamines such as Polyquarto H sold by Henkel under the reference name “Polyethylene glycol (15) Tallow polyamine” in the CTFA dictionary;

(14) crosslinked (meth)acryloyloxy(C₁-C₄)alkyltri-(C₁-C₄)alkylammonium salt polymers such as those sold under the name Salcare® SC 92, Salcare® SC 95 and Salcare® SC 96 by the company Allied Colloids; and mixtures thereof.

Other cationic polymers that may be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives.

The amphoteric polymers that may be used in accordance with the invention may be chosen from polymers comprising units B and C randomly distributed in the polymer chain, in which B denotes a unit derived from a monomer comprising at least one basic nitrogen atom and C denotes a unit derived from an acidic monomer comprising one or more carboxylic or sulphonic groups, or alternatively B and C may denote groups derived from zwitterionic carboxybetaine or sulphobetaine monomers; B and C may also denote a cationic polymer chain comprising primary, secondary, tertiary or quaternary amine groups, in which at least one of the amine groups bears a carboxylic or sulphonic group linked via a hydrocarbon-based radical, or alternatively B and C form part of a chain of a polymer containing an ethylene-dicarboxylic unit in which one of the carboxylic groups has been made to react with a polyamine comprising one or more primary or secondary amine groups.

The amphoteric polymers corresponding to the above definition that are more particularly preferred are chosen from the following polymers:

(1) polymers resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylate and acrylate, dialkylaminoalkylmethacrylamide and -acrylamide. Such compounds are described in U.S. Pat. No. 3,836,537. Mention may also be made of the sodium acrylate/acrylamidopropyltrimethylammonium chloride copolymer sold under the name Polyquart® KE 3033 by the company Henkel.

The vinyl compound may also be a dialkyldiallylammonium salt such as diethyldiallylammonium chloride.

The copolymers of acrylic acid and of the latter monomer are sold under the names Merquat® 280, Merquat® 295 and Merquat® Plus 3330 by the company Calgon.

(2) Polymers containing units derived from:

a) at least one monomer chosen from acrylamides and methacrylamides substituted on the nitrogen with an alkyl radical,

b) at least one acidic comonomer containing one or more reactive carboxylic groups, and

c) at least one basic comonomer such as esters containing primary, secondary, tertiary and quaternary amine substituents of acrylic and methacrylic acids and the product of quaternization of dimethylaminoethyl methacrylate with dimethyl or diethyl sulphate.

The N-substituted acrylamides or methacrylamides which are more particularly preferred according to the invention are groups in which the alkyl radicals contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen more particularly from acrylic acid, methacrylic acid, crotonic acid, itaconic acid, maleic acid and fumaric acid and alkyl monoesters, having 1 to 4 carbon atoms, of maleic or fumaric acids or anhydrides.

The preferred basic comonomers are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer such as the products sold under the name Amphomer® or Lovocryl® 47 by the company National Starch are particularly used.

(3) Crosslinked and alkylated polyamino amides partially or totally derived from polyamino amides of general formula:

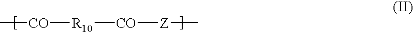

in which R₁₀ represents a divalent radical derived from a saturated dicarboxylic acid, a mono- or dicarboxylic aliphatic acid containing an ethylenic double bond, an ester of a lower alkanol, having 1 to 6 carbon atoms, of these acids or a radical derived from the addition of any one of the said acids to a bis(primary) or bis(secondary) amine, and Z denotes a bis(primary), mono- or bis(secondary) polyalkylene-polyamine radical and preferably represents:

a) in proportions of from 60 to 100 mol %, the radical

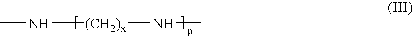

where x=2 and p=2 or 3, or alternatively x=3 and p=2 this radical being derived from diethylenetriamine, from triethylenetetraamine or from dipropylenetriamine;

b) in proportions of from 0 to 40 mol %, the radical (III) above in which x=2 and p=1 and which is derived from ethylenediamine, or the radical derived from piperazine:

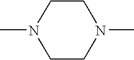

c) in proportions of from 0 to 20 mol %, the —NH—(CH₂)₆—NH— radical derived from hexamethylenediamine, these polyamino amines being crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid and acids containing an ethylenic double bond such as, for example, acrylic acid, methacrylic acid and itaconic acid. The alkane sultones used in the alkylation are preferably propane sultone or butane sultone, and the salts of the alkylating agents are preferably the sodium or potassium salts.

(4) Polymers containing zwitterionic units of formula:

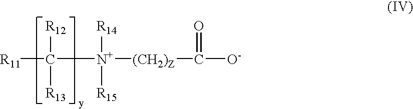

in which R₁₁ denotes a polymerizable unsaturated group such as an acrylate, methacrylate, acrylamide or methacrylamide group, y and z each represent an integer from 1 to 3, R₁₂ and R₁₃ represent a hydrogen atom, a methyl, ethyl or propyl group, R₁₄ and R₁₅ represent a hydrogen atom or an alkyl radical such that the sum of the carbon atoms in R₁₄ and R₁₅ does not exceed 10.

The polymers comprising such units can also contain units derived from non-zwitterionic monomers such as dimethyl or diethylaminoethyl acrylate or methacrylate or alkyl acrylates or methacrylates, acrylamides or methacrylamides or vinyl acetate.

By way of example, mention may be made of the copolymer of methyl methacrylate/dimethyl carboxymethylammonio ethyl methacrylate such as the product sold under the name Diaformer® Z301 by the company Sandoz.

(5) Polymers derived from chitosan containing monomer units corresponding to the following formulae:

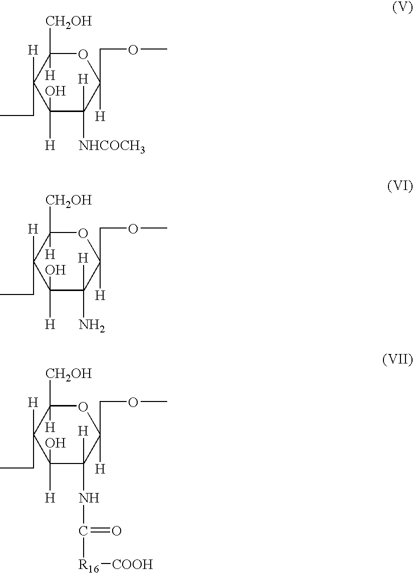

the unit (V) being present in proportions of between 0 and 30%, the unit (VI) in proportions of between 5 and 50% and the unit (VII) in proportions of between 30 and 90%, it being understood that, in this unit F, R₁₆ represents a radical of formula:

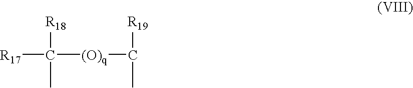

in which if q=0, R₁₇, R₁₈ and R₁₉, which may be identical or different, each represent a hydrogen atom, a methyl, hydroxyl, acetoxy or amino residue, a monoalkylamine residue or a dialkylamine residue which are optionally interrupted by one or more nitrogen atoms and/or optionally substituted with one or more amine, hydroxyl, carboxyl, alkylthio or sulphonic radicals, an alkylthio residue in which the alkyl radical bears an amino residue, at least one of the radicals R₁₇, R₁₈ and R₁₉ being, in this case, a hydrogen atom;

or, if q=1, R₁₇, R₁₈ and R₁₉ each represent a hydrogen atom, as well as the salts formed by these compounds with bases or acids.

(6) Polymers derived from the N-carboxyalkylation of chitosan, such as N-carboxymethylchitosan or N-carboxybutylchitosan sold under the name “Evalsan®” by the company Jan Dekker.

(7) Polymers corresponding to the general formula (IX) as described, for example, in French patent 1 400 366:

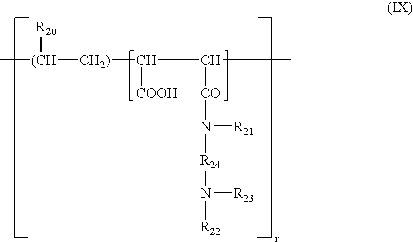

in which R₂₀ represents a hydrogen atom, a CH₃O, CH₃CH₂O or phenyl radical, R₂₁ denotes hydrogen or a lower alkyl radical such as methyl or ethyl, R₂₂ denotes hydrogen or a lower alkyl radical such as methyl or ethyl, R₂₃ denotes a lower alkyl radical such as methyl or ethyl or a radical corresponding to the formula: —R₂₄—N(R₂₂)₂, R₂₄ representing a —CH₂—CH₂—, —CH₂—CH₂—CH₂— or —CH₂—CH(CH₃)— group, R₂₂ having the meanings mentioned above,

as well as the higher homologues of these radicals and containing up to 6 carbon atoms.

(8) Amphoteric polymers of the type -D-X-D-X— chosen from:

a) polymers obtained by the action of chloroacetic acid or sodium chloroacetate on compounds containing at least one unit of formula:

-D-X-D-X-D- (X)

where D denotes a radical

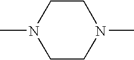

and X denotes the symbol E or E′, E or E′, which may be identical or different, denotes a divalent radical which is an alkylene radical with a straight or branched chain containing up to 7 carbon atoms in the main chain, which is unsubstituted or substituted with hydroxyl groups and which can contain, in addition to the oxygen, nitrogen and sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups;

b) polymers of formula:

-D-X-D-X- (XI)

where D denotes a radical

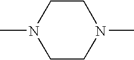

and X denotes the symbol E or E′ and at least once E′; E having the meaning given above and E′ being a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily containing one or more carboxyl functions or one or more hydroxyl functions and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also contain other vinyl comonomers such as vinylcaprolactam.

The silicones that may be used in accordance with the invention may be soluble or insoluble in water, and they may be in particular polyorganosiloxanes that are insoluble in water; they may be in the form of oils, waxes, resins or gums.

The organopolysiloxanes are defined in greater detail in Walter Noll's “Chemistry and Technology of Silicones” (1968) Academic Press. They can be volatile or non-volatile.

When they are volatile, the silicones are more particularly chosen from those having a boiling point of between 60° C. and 260° C., and even more particularly from:

(i) cyclic silicones containing from 3 to 7 and preferably from 4 to 5 silicon atoms. These are, for example, octamethylcyclotetrasiloxane sold in particular under the name “Volatile Silicone 7207” by Union Carbide or “Silbione 70045 V 2” by Rhodia, decamethylcyclopentasiloxane sold under the name “Volatile Silicone 7158” by Union Carbide, and “Silbione 70045 V 5” by Rhodia.

Mention may also be made of cyclocopolymers of the dimethylsiloxane/methylalkylsiloxane type, such as “Volatile Silicone FZ 3109” sold by the company Union Carbide, having the chemical structure:

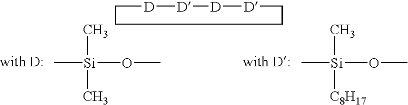

(ii) linear volatile silicones containing 2 to 9 silicon atoms and having a viscosity of less than or equal to 5×10⁻⁶ m²/s at 25° C. An example is decamethyltetrasiloxane sold in particular under the name “SH 200” by the company Toray Silicone. Silicones belonging to this category are also described in the article published in Cosmetics and Toiletries, Vol 91, January 76, pp. 27-32, Todd & Byers “Volatile Silicone Fluids for Cosmetics”.

Among the non-volatile silicones that may be mentioned especially are polyalkylsiloxanes, polyarylsiloxanes, polyalkylarylsiloxanes, silicone gums and resins, and polyorganosiloxanes modified with organofunctional groups.

The organomodified silicones that may be used in accordance with the invention are silicones as defined above and containing in their structure one or more organofunctional groups attached via a hydrocarbon-based group.

Among the organomodified silicones that may be mentioned are polyorganosiloxanes containing:

polyethyleneoxy and/or polypropyleneoxy groups optionally containing C₆-C₂₄ alkyl groups, such as the products known as dimethicone copolyol sold by the company Dow Corning under the name DC 1248 or the oils Silwet® L 722, L 7500, L 77 and L 711 from the company Union Carbide and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning The substituted amine groups are, in particular, C₁-C₄ aminoalkyl groups;

thiol groups such as the products sold under the names “GP 72 A” and “GP 71” from Genesee;

alkoxylated groups such as the product sold under the name “Silicone Copolymer F-755” by SWS Silicones and Abil Wax® 2428, 2434 and 2440 by the company Goldschmidt;

hydroxylated groups such as the polyorganosiloxanes containing a hydroxyalkyl function, described in French patent application FR-A-85/16334;

acyloxyalkyl groups such as, for example, the polyorganosiloxanes described in patent U.S. Pat. No. 4,957,732;

anionic groups of the carboxylic acid type, such as, for example, in the products described in patent EP 186 507 from the company Chisso Corporation, or of alkylcarboxylic type, such as those present in the product X-22-3701E from the company Shin-Etsu; 2-hydroxyalkyl sulphonate; 2-hydroxyalkyl thiosulphate such as the products sold by the company Goldschmidt under the names “Abil® S201” and “Abil® S255 ”.

hydroxyacylamino groups, such as the polyorganosiloxanes described in patent application EP 342 834. Mention may be made, for example, of the product Q2-8413 from the company Dow Corning.

Mention may be made especially, as oils of plant origin, of sweet almond oil, avocado oil, castor oil, olive oil, jojoba oil, sunflower oil, wheatgerm oil, sesame seed oil, groundnut oil, grapeseed oil, soybean oil, rapeseed oil, safflower oil, coconut oil, corn oil, hazelnut oil, carite butter, palm oil, apricot kernel oil or beauty-leaf oil; as an oil of animal origin, of perhydrosqualene; as oils of mineral origin, of liquid paraffin and liquid petroleum jelly.

The polyisobutenes and poly(α-olefins) are chosen from those that are well known in the art.

Examples of esters that may especially be mentioned are fatty acid esters, for instance isopropyl myristate, isopropyl palmitate, 2-ethylhexyl palmitate, purcellin oil (stearyl octanoate), isononyl isononanoate, isostearyl isononanoate and isopropyl lanolate, and mixtures thereof.

The anionic polymers generally used in the present invention are polymers comprising groups derived from carboxylic acids, sulphonic acids or phosphoric acids, and having a weight-average molecular mass of between 500 and 5,000,000.

The carboxylic groups are provided by unsaturated monocarboxylic or dicarboxylic acid monomers such as those corresponding to the formula:

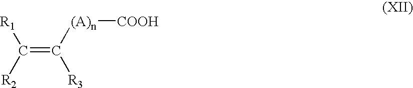

in which n is an integer from 0 to 10, A denotes a methylene group, optionally connected to the carbon atom of the unsaturated group or to the neighbouring methylene group when n is greater than 1 via a hetero atom such as oxygen or sulphur, R₁ denotes a hydrogen atom or a phenyl or benzyl group, R₂ denotes a hydrogen atom or a lower alkyl or carboxyl group, and R₃ denotes a. hydrogen atom, a lower alkyl group or a —CH₂—COOH, phenyl or benzyl group.

In formula (XII) above, a lower alkyl group preferably contains from 1 to 4 carbon atoms and in particular denotes methyl and ethyl groups.

The anionic polymers containing carboxylic groups which are preferred according to the invention are:

A) homo- or copolymers of acrylic or methacrylic acid or salts thereof and in particular the products sold under the names Versicolo E or K by the company Allied Colloid, Ultraholdo by the company BASF. The copolymers of acrylic acid and acrylamide sold in the form of their sodium salt under the names Reten® 09 421, 423 or 425 by the company Hercules, and the sodium salts of polyhydroxycarboxylic acids;

B) copolymers of acrylic or methacrylic acids with a monoethylenic monomer such as ethylene, styrene, vinyl esters and acrylic or methacrylic acid esters. These copolymers are optionally grafted onto a polyalkylene glycol such as polyethylene glycol and optionally crosslinked. Such polymers are described in particular in French patent 1 222 944 and German patent application No. 2 330 956. Mention may be made in particular of copolymers whose chain comprises an optionally N-alkylated and/or hydroxyalkylated acrylamide unit, such as those described in particular in the Luxembourg patent applications 75370 and 75371 or sold under the name Quadramer® by the company American Cyanamid. Mention may also be made of copolymers of acrylic acid and of C₁-C₄ alkyl methacrylate and the copolymer of methacrylic acid and of ethyl acrylate sold under the name Luvimer® MAEX by the company BASF;

C) copolymers derived from crotonic acid, such as those whose chain comprises vinyl acetate or propionate units and optionally other monomers such as allylic or methallylic esters, vinyl ether or vinyl ester of a saturated, linear or branched carboxylic acid containing a long hydrocarbon-based chain such as those comprising at least 5 carbon atoms, it being possible for these polymers to be grafted and crosslinked, or alternatively a vinyl, allylic or methallylic ester of an α- or β-cyclic carboxylic acid. Such polymers are described, inter alia, in French patents 1 222 944, 1 580 545, 2 265 782, 2 265 781, 1 564 110 and 2 439 798. Commercial products falling within this category are the resins 28-29-30, 26-13-14 and 28-13-10 sold by the company National Starch;

D) polymers derived from maleic, fumaric or itaconic acids or anhydrides with vinyl esters, vinyl ethers, vinyl halides, phenylvinyl derivatives or acrylic acid and its esters; these polymers may be esterified. Such polymers are described in particular in U.S. Pat. Nos. 2,047,398, 2,723,248 and 2,102,113 and GB patent 839 805, and especially those sold under the names Gantrez® AN or ES by the company ISP.

Polymers also falling within this category are the copolymers of maleic, citraconic or itaconic anhydrides and of an allylic or methallylic ester optionally comprising an acrylamide or methacrylamide group, an α-olefin, acrylic or methacrylic esters, acrylic or methacrylic acid or vinylpyrrolidone in their chain, the anhydride functions being monoesterified or monoamidated. These polymers are described, for example, in French patents 2 350 384 and 2 357 241 by the Applicant;

E) polyacrylamides comprising carboxylate groups.

The polymers comprising sulphonic groups are polymers comprising vinylsulphonic, styrenesulphonic, naphthalenesulphonic or acrylamidoalkylsulphonic units.

These polymers can be chosen in particular from:

polyvinylsulphonic acid salts with a molecular weight of between about 1 000 and 100 000, as well as copolymers with an unsaturated comonomer such as acrylic or methacrylic acids and esters thereof, as well as acrylamide or derivatives thereof, vinyl ethers and vinylpyrrolidone;

polystyrenesulphonic acid salts, the sodium salts having a molecular weight of about 500 000 and of about 100 000, sold, respectively, under the names Flexan® 500 and Flexan® 130 by National Starch. These compounds are described in patent FR 2 198 719;

polyacrylamidesulphonic acid salts such as those mentioned in U.S. Pat. No. 4,128,631 and more particularly polyacrylamidoethylpropanesulphonic acid sold under the name Cosmedia Polymer® HSP 1180 by Henkel.

According to the invention, the anionic polymers are preferably chosen from acrylic acid copolymers such as the acrylic acid/ethyl acrylate/N-tert-butylacrylamide terpolymer sold under the name Ultrahold Strong® by the company BASF, copolymers derived from crotonic acid, such as the vinyl acetate/vinyl tert-butyl-benzoate/crotonic acid terpolymers and the crotonic acid/vinyl acetate/vinyl neododecanoate terpolymers sold under the name Resin 28-29-30 by the company National Starch, polymers derived from maleic, fumaric or itaconic acids with vinyl esters, vinyl ethers, vinyl halides, phenylvinyl derivatives or acrylic acid and esters thereof, such as the methyl vinyl ether/monoesterified maleic anhydride copolymer sold under the name Gantrez® ES 425 by the company ISP, the copolymers of methacrylic acid and of methyl methacrylate sold under the name Eudragit® L by the company Rohm Pharma, the copolymer of methacrylic acid and of ethyl acrylate sold under the name Luvimer® MAEX by the company BASF, the vinyl acetate/crotonic acid copolymer sold under the name Luviset® CA 66 by the company BASF and the vinyl acetate/crotonic acid/polyethylene glycol terpolymer sold under the name Aristoflex® A by the company BASF.

According to the invention, the anionic polymers may also be used in the form of a latex or pseudolatex, i.e. in the form of an aqueous dispersion of insoluble polymer particles.

As nonionic polymers that may be used according to the present invention, mention may be made especially of:

vinylpyrrolidone homopolymers;

copolymers of vinylpyrrolidone and vinyl acetate;

polyalkyloxazolines such as the polyethyloxazolines sold by the company Dow Chemical under the names Peox® 50 000, Peox® 200 000 and Peox® 500 000;

vinyl acetate homopolymers, such as the product sold under the name Appretan® EM by the company Hoechst, or the product sold under the name Rhodopas® A 012 by the company Rhône-Poulenc;

copolymers of vinyl acetate and acrylic ester, such as the product sold under the name Rhodopas® AD 310 by Rhônne-Poulenc;

copolymers of vinyl acetate and ethylene, such as the product sold under the name Appretan® TV by the company Hoechst;

copolymers of vinyl acetate and maleic ester, for example of dibutyl maleate, such as the product sold under the name Appretan® MB Extra by the company Hoechst;

copolymers of polyethylene and maleic anhydride;

alkyl acrylate homopolymers and alkyl methacrylate homopolymers, such as the product sold under the name Micropearl® RQ 750 by the company Matsumoto or the product sold under the name Luhydran® A 848 S by the company BASF;

acrylic ester copolymers such as, for example, copolymers of alkyl acrylates and alkyl methacrylates, such as the products sold by the company Rohm & Haas under the names Primal® AC-261 K and Eudragit® NE 30 D, by the company BASF under the names Acronal® 601, Luhydran® LR 8833 or 8845, and by the company Hoechst under the names Appretan® N 9213 or N 921 2;

copolymers of acrylonitrile and a nonionic monomer chosen, for example, from butadiene and alkyl (meth)acrylates; mention may be made of the products sold under the names Nipol® LX 531 8 by the company Nippon Zeon or those sold under the name CJ 0601 8 by the company Rohm & Haas;

polyurethanes, such as the products sold under the names Acrysol® RM 1020 or Acrysol® RM 2020 by the company Rohm & Haas, and the products Uraflex® XP 401 UZ and Uraflex® XP 402 UZ by the company DSM Resins;

copolymers of alkyl acetate and urethane, such as the product 8538-33 by the company National Starch;

polyamides, such as the product Estapor® LO 11 sold by the company Rhône-Poulenc;

unmodified or chemically modified nonionic guar gums.

The unmodified nonionic guar gums are, for example, the products sold under the name Vidogum® GH 175 by the company Unipectine and under the name Jaguar® C by the company Meyhall.

The modified nonionic guar gums that can be used according to the invention are preferably modified with C₁-C₆ hydroxyalkyl groups. Mention may be made, for example, of hydroxymethyl, hydroxyethyl, hydroxypropyl and hydroxybutyl groups.

These guar gums are well known in the state of the art and can be prepared, for example, by reacting corresponding alkene oxides, such as, for example, propylene oxides, with guar gum so as to obtain a guar gum modified with hydroxypropyl groups.

Such nonionic guar gums optionally modified with hydroxyalkyl groups are sold, for example, under the trade names Jaguar® HP8, Jaguar® HP60 and Jaguar® HP120, Jaguar® DC 293 and Jaguar® HP 105 by the company Meyhall or under the name Galactasol® 4H4FD2 by the company Aqualon.

The alkyl groups of the nonionic polymers preferably contain from 1 to 6 carbon atoms.

The reducing agents may be chosen from thio acids and salts thereof (thioglycolic acid or thiolactic acid, cysteine or cysteamine), alkali metal sulphites or alkaline-earth metal sulphites, reducing sugars such as glucose, vitamin C and its derivatives, sulphinic acid derivatives and phosphines.

The composition according to the invention may contain one or more of the biotin compounds and complexing agents described above, in a total amount of between 0.001% and 90% by weight, preferably between 0.01% and 25% by weight and better still between 0.1 and 20% by weight, relative to the total weight of the cosmetic treatment composition.

The cosmetically acceptable medium may consist solely of water, or of a solvent or mixture of solvents, or of a mixture of water and one or more cosmetically acceptable solvents such as a C₁-C₄ lower alcohol, for instance ethanol, isopropanol, tert-butanol or n-butanol; alkylene glycols, for instance propylene glycol, and glycol ethers.

The pH of the compositions according to the invention is between 4 and 8 and preferably between 5 and 7.

The compositions according to the invention may also contain additives such as natural or synthetic, anionic, amphoteric, zwitterionic, nonionic or cationic, associative or non-associative polymeric thickeners, non-polymeric thickeners, for instance acids or electrolytes, anionic, nonionic or amphoteric surfactants, nacreous agents, opacifiers, fragrances, colorants, mineral or organic particles, preserving agents and pH stabilizers.

A person skilled in the art will take care to select the optional additives and the amount thereof such that they do not harm the properties of the treatment compositions.

These additives are present in the composition according to the invention in an amount ranging from 0 to 20% by weight relative to the total weight of the composition.

The compounds b) are preferably chosen from compounds of structure:

D(A′)_{n′}(X)ₘ (II)

in which m denotes 0 or 1 and X denotes Z′ or CW′;

Z′, W′, A′ and n′ having the same possible meanings as Z, W, A and n, respectively, in formula (I) and C being a species for fixing the residue W to the active complexation element D, D being such that the combination D(A′)_{n′}(X)ₘ can form with the compound(s) a) a complex whose dissociation constant is less than 10⁻¹ at a temperature of 25° C. For example, D(A′)_{n′}(X)ₘ denotes avidin (n′=m=0) or streptavidin (n′=m=0) or antibiotin antibodies and fragments thereof (n′=m=0) or avidin-alkaline phosphatase (n′=0, m=1) or avidin-peroxidase (n′=0, m=1).

These compositions may be in the form of more or less thick solutions, creams, fluid gels, sticks or mousses. They may be packaged in aerosol form.

One subject of the invention is a set of processes for the cosmetic treatment of keratin materials, and in particular the hair.

A first process according to the invention consists, in a first stage, in depositing on the hair by adsorption onto the keratin materials a compound b) in which m=0 or m=1, X denoting Z′ if m⁻¹, and then in a second stage in applying a compound a) in which Y denotes BW.

A second process according to the invention consists, in a first stage, in depositing by adsorption onto the keratin materials a compound b) in which X denotes CW′ and m=1, and then, in a second stage, in applying a compound a) in which Y denotes BW.

A third process according to the invention consists, in a first stage, in depositing on the keratin materials by adsorption a compound a) in which Y denotes Z, and then, in a second stage, in applying a compound b) in which X denotes CW′ and m=1.

A fourth process according to the invention consists, in a first stage, in depositing on the keratin materials by adsorption a compound a) in which Y denotes BW, and then, in a second stage, in applying a compound b) in which m=0 or m=1 with X denoting Z′ if m=1.

A fifth process according to the invention consists, in a first stage, in simultaneously depositing on the keratin materials by adsorption a compound a) in which Y denotes BW, and then, in a second stage, in applying a compound b) in which X=CW′ and m=1.

A sixth process according to the invention consists in simultaneously depositing on the keratin materials by adsorption a compound a) in which Y denotes Z and a compound b) in which X=CW′ and m=1.

A seventh process according to the invention consists in simultaneously depositing on the keratin materials by adsorption a compound a) in which Y denotes BW and a compound b) in which m=0 or m=1 with X denoting Z′ if m=1.

An eighth process according to the invention consists in simultaneously depositing on the keratin materials by adsorption a compound a) in which Y denotes BW and a compound b) in which m=1 and X=CW′.

Optionally, for all the processes, it is possible to provide one or more compounds a) or b) in one or more additional steps. Multilayer deposits may thus be formed.

Another subject of the invention consists of multi-compartment kits. A first kit contains, in a first compartment, a compound a) in which Y=Z and, in a second compartment, a compound b) in which X=CW′ and m=1.

A second kit contains, in a first compartment, a compound a) in which Y=BW and, in a second compartment, a compound b) in which m=0 or m=1, X denoting Z′ if m=1.

A third kit contains, in a first compartment, a compound a) in which Y=BW and, in a second compartment, a compound b) in which m=1 and X=CW′.

The hair treatment process according to the invention may comprise a preliminary step that consists of a cosmetic treatment chosen from the group formed by reducing agents or oxidizing agents for permanently reshaping the hair, oxidation dyes, shampoos or styling compositions.

The examples that follow are intended to illustrate the invention without, however, being limiting in nature.

### EXAMPLE 1

In order to demonstrate the remanence properties of the invention compared with a simple physicochemical adsorption, gold nanoparticles were deposited on hair fibres with or without avidin in the medium, biotin being fixed to the particles to be deposited.

1) Particles Deposited With Biotin and Avidin (Sample 1)

30 mg of hair (i.e. about 20 hairs) are taken from a lock of prewashed natural hair and placed in a small glass flask.

A solution of avidin (Sigma-Aldrich A9275) at a concentration of 1.5 mg/ml in a phosphate buffer solution (pH 7.2; 0.15 M) is prepared. 3 ml of this avidin solution are placed in contact with the hairs to be treated, while bubbling nitrogen through the solution at room temperature, until evaporation is complete.

Rinsing is then performed in several steps:

rinsing with 3 ml of a phosphate buffer solution+Tween 20 at 0.05%, for 1 minute 30 seconds,

rinsing with 3 ml of a phosphate buffer solution+Tween 20 at 0.05%, for 30 seconds,

four rinses with 5 ml of distilled water, for 30 seconds.

The hair is then dried with the gentle heat of a hairdryer (about 30° C.). 5 of these hairs treated with avidin are placed in a glass flask. 100 μl of a solution containing gold particles (20 nm) in suspension (Sigma; albumin-biotin 20 nm gold labelled) are added to 700 μl of a phosphate buffer solution containing Tween 20 at 0.05% and BSA (bovine serum albumin) at 0.5%. This solution is placed in contact with the 5 hairs pretreated with avidin and the system is incubated for 24 hours, with stirring and at room temperature.

Rinsing is performed in several steps:

rinsing with 2 ml of a phosphate buffer solution, 0.05% Tween and 0.5% BSA, for 30 seconds,

rinsing with 2 ml of a phosphate buffer solution for 1 minute,

3 rinses with 2 ml of distilled water, for 1 minute 30 seconds.

2) Particles Deposited with Biotin and Without Avidin (Sample 2)

5 hairs are taken from a lock of prewashed natural hair and placed in a glass flask, to which are added 500 μl of a phosphate buffer solution (pH 7.2; 0.15 M). The system is incubated for 24 hours with stirring and at room temperature. After removing the supernatant, the hairs are rinsed 3 times with distilled water for 1 minute 30 seconds. They are dried with gentle heat using a hairdryer (about 30° C.).

100 μl of a solution containing gold particles (20 nm) in suspension (Sigma; albumin—biotin—20 nm gold labelled) are added to 700 μl of a phosphate buffer solution containing 0.05% Tween 20 and 0.5% BSA. This solution is placed in contact with the 5 control hairs and the system is incubated for 24 hours with stirring and at room temperature.

The same rinsing operation as described previously is performed.

The hairs are then analysed by Scanning Electron Microscopy and EDX (Energy Dispersive X-ray).

After several washes, the sample treated with the avidin/biotin system (sample 1) still shows the presence of gold nanoparticles on the surface. There is thus pronounced remanence after washing. On the other hand, no gold nanoparticles are detected on the hair not treated with avidin (sample 2).

This example clearly shows the value of the avidin/biotin ligand-receptor couple for creating a deposit that is wash-remanent.

### Example 2

### Wash-Remanent Grafting of an Enzyme (Peroxidase) by Means of the Biotin/Avidin Couple

Adsorption of Biotin-Derivatized Peroxidase onto Hair Treated with Avidin (Sample 1):

30 mg of hair (i.e. about 20 hairs) are taken from a lock of prewashed natural grey hair and placed in a small glass flask. A solution of avidin (Sigma-Aldrich A9275) at 1.5 mg/ml in a phosphate buffer solution (pH 7.2; 0.15 M) is prepared. 3 ml of this avidin solution are placed in contact with the hairs to be treated for 24 hours at room temperature.

Rinsing is then performed in several steps:

1. rinsing with 3 ml of a phosphate buffer solution+0.05% Tween 20, for 1 minute 30 seconds,

2. rinsing with 3 ml of a phosphate buffer solution+0.05% Tween 20, for 30 seconds,

four rinses with 5 ml of distilled water, for 30 seconds.

These hairs treated with avidin are then dried with gentle heat using a hairdryer (about 30° C.) for about 20 minutes.

A solution of biotinylated peroxidase (reference P 9568, sold by Sigma) at 0.1 mg/ml in phosphate buffer is prepared. The biotinylated peroxidase solution is diluted tenfold. 1 ml of this solution is placed in contact with the samples (5 hairs) for 1 hour at room temperature. The hairs are then rinsed with 2 ml of distilled water for 1 minute, three times consecutively.

To demonstrate the peroxidase fixed to the hair, a kit sold by Sigma (AEC-101 Staining Kit) is used. 1 ml of substrate is poured into each flask and left in contact with the hairs for exactly 5 minutes. The samples are rinsed 3 times with 2 ml of distilled water. Two hairs of each sample are removed with tweezers and mounted on a glass slide.

Adsorption of Biotin-Derivatized Peroxidase onto Hair not Treated with Avidin (Sample 2):

The same protocol as described previously is performed on hairs not treated with avidin.

After these experiments, the hairs are analysed along their entire length by optical microscopy in white light.

After washing several times, the sample treated with the avidin/biotin system (sample 1) still shows the presence of peroxidase (red coloration) on the surface. There is thus pronounced remanence after washing. On the other hand, no peroxidase (absence of red coloration) is detected on the hair not treated with avidin (sample 2)

1. Cosmetic composition comprising:
a biotin compound a) and an agent b) capable of forming a complex, with a dissociation constant of less than or equal to 10⁻¹ at a temperature of 25° C., with the biotin compound, the biotin compound or the complexing agent bearing at least one group with non-colouring cosmetic activity.

2. Composition according to claim 1, characterized in that compound a) is chosen from the compounds of formula (I) 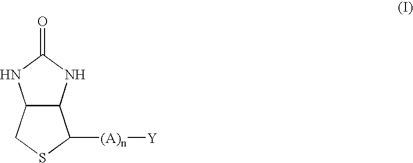
with A denoting a linear or branched, saturated or unsaturated divalent radical containing from 1 to 100 carbon atoms, optionally interrupted with one or more hetero atoms and optionally substituted with one or more groups chosen from hydroxyl, amino, halogen, aryl, phosphate, phosphorate, sulphate, sulphonate, carboxyl, alkoxycarbonyl and alkoxy groups,
with Y denoting a group Z chosen from carboxyl, mercaptan, alkoxycarbonyl, amino, ammonio or a radical BW, W being a residue with cosmetic activity and B being a species for fixing the residue W to the biotin-based skeleton,
with n denoting 0 or 1,
with B denoting, for example, an amine, imine, amide, ester, disulphide, thioester, urethane, urea, ether, thioether, azo, methine or epoxy group, an aromatic or heterocyclic residue or a dialkylpolysiloxane residue,
with W possibly being derived from UV-screening molecules, hydrating or emollient molecules, conditioners, antistatic agents, antiperspirants, fragrancing materials, reducing molecules, oxidizing molecules, antimicrobial agents, antidandruff agents, mineral or organic particulates onto which are optionally absorbed one or more polymers, and anionic or nonionic, amphoteric or cationic film-forming agents onto which are optionally adsorbed organic or mineral particles.

3. Composition according to either of claims 1 and 2, characterized in that compound b) is chosen from compounds of structure:
D(A′)_{n′}(X)ₘ (II)
in which m denotes 0 or 1 and X denotes Z′ or CW′;
Z′, W′, A′ and n′ having the same possible meanings as Z, W, A and n, respectively, in formula (I)
C being a species for fixing the residue W to the active complexation element D,
D being such that the combination D(A′)_{n′}(X)ₘ can form with the compound(s) a) a complex whose dissociation constant is less than 10⁻¹ at a temperature of 25° C.

4. Composition according to any one of claims 1 to 3, characterized in that compound b) denotes avidin.

5. Composition according to any one of claims 1 to 3, characterized in that compound b) denotes streptavidin.

6. Composition according to any one of claims 1 to 3, characterized in that compound b) denotes an antibiotin antibody.

7. Composition according to any one of claims 1 to 6, characterized in that it is in the form of a more or less thick aqueous, alcoholic or aqueous-alcoholic solution, a cream, a fluid gel, a stick or a mousse, optionally packaged in aerosol form.

8. Cosmetic process for treating keratin materials, and in particular the hair, which consists in depositing on the keratin materials by adsorption, in a first stage, a compound b) in which m=0 or m=1, X denoting Z′ if m=1, and then, in a second stage, in applying a compound a) in which Y denotes BW.

9. Cosmetic process for treating keratin materials, and in particular the hair, which consists in depositing on the keratin materials by adsorption, in a first stage, a compound b) in which X denotes CW′ and m=1, and then, in a second stage, in applying a compound a) in which Y denotes BW.

10. Cosmetic process for treating keratin materials, and in particular the hair, which consists in depositing on the keratin materials by adsorption, a compound a) in which Y denotes Z, and then, in a second stage, in applying a compound b) in which X denotes CW′ and m=1.

11. Cosmetic process for treating keratin materials, and in particular the hair, which consists in depositing on the keratin materials by adsorption, a compound a) in which Y denotes BW, and then, in a second stage, in applying a compound b) in which m=0 or m=1 with X denoting Z′ if m=1.

12. Cosmetic process for treating keratin materials, and in particular the hair, which consists in simultaneously depositing on the keratin materials by adsorption, a compound a) in which Y denotes BW, and then, in a second stage, in applying a compound b) in which X=CW′ and m=1.

13. Cosmetic process for treating keratin materials, and in particular the hair, which consists in simultaneously depositing on the keratin materials by adsorption a compound a) in which Y denotes Z and a compound b) in which X=CW′ and m=1.

14. Cosmetic process for treating keratin materials, and in particular the hair, which consists in simultaneously depositing on the keratin materials by adsorption a compound a) in which Y denotes BW and a compound b) in which m=0 or m=1 with X denoting Z′ if m=1.

15. Cosmetic process for treating keratin materials, and in particular the hair, which consists in simultaneously depositing on the keratin materials by adsorption a compound a) in which Y denotes BW and a compound b) in which m=1 and X=CW′.

16. Process for treating keratin materials, characterized in that it comprises a subsequent step in which at least one compound a) or one compound b) is provided.

17. Kit for treating keratin materials, characterized in that it comprises, in a first compartment, a compound a) in which Y=Z and, in a second compartment, a compound b) in which X=CW′ and m=1.

18. Kit for treating keratin materials, characterized in that it comprises, in a first compartment, a compound a) in which Y=BW, in a second compartment, a compound b) in which m=0 or m=1, X denoting Z′ if m=1.

19. Kit for treating keratin materials, characterized in that it comprises, in a first compartment, a compound a) in which Y=BW and, in a second compartment, a compound b) in which m=1 and X=CW′.

20. Process for treating keratin materials, characterized in that it comprises a preliminary step that consists of a cosmetic treatment chosen from the group formed by reducing agents or oxidizing agents for permanently reshaping the hair, oxidation dyes, bleaches, shampoos or styling compositions, and then in that a sufficient amount of a cosmetic composition as defined in any one of claims 1 to 7 is applied.

21. Cosmetic composition comprising a biotin compound a) as defined in claim 3, Y denoting BW.

22. Cosmetic composition comprising a complexing agent b) as defined in claim 4, with m=1 and X denoting CW′.
